(19) United States
(12) Patent Application Publication
(10) Pub. No.: US 2023/0364390 A1
(43) Pub. Date: Nov. 16, 2023

(54) TUBE SECUREMENT DEVICE AND METHODS

(71) Applicant: The Board of Regents of The University of Texas System, Austin, TX (US)

(72) Inventors: Joshua Kless Kern, Dallas, TX (US); Philip Colvin Jarrett, III, Farmers Branch, TX (US)

(21) Appl. No.: 18/196,262

(22) Filed: May 11, 2023

Related U.S. Application Data
(60) Provisional application No. 63/340,905, filed on May 11, 2022.
(60) Provisional application No. 63/387,190, filed on Dec. 13, 2022.

(51) Int. Cl. A61M 25/02

(52) U.S. Cl. CPC A61M 25/02; A61M 2025/0266; A61M 2207/10

(57) ABSTRACT

A surgical tube placement and securement device and methods of use and manufacture. The device has a base flange formed of malleable material and includes a first opening at a first end and a second opening at a second end. The first opening is defined by an inner surface which grips an outer surface of an insertion tube during use. The second opening is defined by a bottom surface that defines a perimeter of the second opening around a target insertion area. The second opening has a diameter greater than the first opening, and secures to a skin of the patient by using a fastener (e.g., an adhesive) disposed on the bottom surface which contacts and adheres to the skin. The insertion tube passes through the first opening and the second opening, is gripped at the first opening, and is inserted into the skin at the target area.

### CROSS-REFERENCE TO RELATED APPLICATIONS

This application claims priority to U.S. Provisional Patent Application No. 63/340,905 titled “TUBE SECUREMENT DEVICE AND METHODS” and filed May 11, 2022, and U.S. Provisional Patent Application No. 63/387,190 titled “TUBE SECUREMENT DEVICE AND METHODS” filed Dec. 13, 2022, the contents of which are incorporated herein by reference their entireties.

### BACKGROUND

### 1. Field

The presently disclosed technology generally relates to the field of surgical tubes, and more particularly, to a surgical tube placement and securement device and methods of use and manufacture.

### 2. Discussion of Related Art

In medicine, a broad range of tubes are utilized to drain body cavities of air or fluids. Common examples include chest tubes, suprapubic catheters, cholecystostomy tubes and percutaneous nephrostomy tubes. Securing tubes at the skin is necessary to prevent unintentional dislodgment or advancement. However, when a chest tube is secured to the skin of a patient, the suture often fails to adequately secure the tube in place, commonly resulting in tube migration or dislodgement. The suture also fails to achieve fluid occlusion, commonly resulting in air leaks around the tube if not effectively dressed with xeroform gauze and fluid-occlusive tape. The market lacks a simple device capable of effectively securing the broad range of tubes utilized in medicine with adequate adhesion and fluid occlusion.

It is with these observations in mind, among others, that various aspects of the present disclosure were conceived and developed.

### BRIEF SUMMARY

In some scenarios, the systems, methods, and devices disclosed herein address the aforementioned problems. For instance, a device operable to secure a surgical tube to a patient can include at least one of a first portion or a second portion having: an opening at a first end, the opening operable to receive the surgical tube and friction-grip the surgical tube. The device can also include one or more slits formed into a side of at least one of the first portion or the second portion. An adhesive can be disposed on a bottom surface at a second end of at least one of the first portion or the second portion, and the adhesive can be operable to adhere the bottom surface to skin of the patient substantially surrounding a tube insertion area of the surgical tube into the patient.

In some examples, at least one of the first portion or the second portion are formed of a malleable material. The first portion can be a bottom piece of the device and the second portion can be a top piece of the device that mates with the bottom piece. The device can further include a first slit disposed on the bottom piece and a second slit disposed on the top piece configured to align in a different direction than the first slit when the top piece mates with the bottom piece. The first slit can be directed 180° or 90° relative to the second slit. The device can further have one or more curved portions including a first curved portion directed concavely toward an interior of a base flange and forming a transition between a sidewall of the base flange and a second curved portion of the base flange, and/or one or more surface protrusions including an elongated protrusion at the second curved portion. Moreover, the device can have one or more protrusions extending from the second portion configured to mate with a side of the first portion. The device can include an opening neck extending from a planar surface of the second portion. Furthermore, the device can have an upper portion including a first opening, and/or a lower portion including a second opening. The first opening can be narrower than at least a portion of the second opening.

In some instances, a method of using a device operable to secure a surgical tube to a patient includes securing an inner surface of a first opening, formed in a base flange, to a surgical tube, the first opening is operable to receive the surgical tube. Additionally or alternatively, the method can include securing a bottom surface forming a perimeter of a second opening of the base flange to skin of the patient causing the base flange to form the perimeter around a tube insertion area of the surgical tube extending into the patient, wherein an adhesive can be disposed on the bottom surface of the base flange operable to adhere the bottom surface to the skin of the patient.

In some examples, the first opening is at a first end of the base flange and the second opening is at a second end of the base flange, opposite the first end. The second opening can have a diameter greater than the first opening. Additionally, securing the inner surface of the first opening to the surgical tube can include using a friction-fit to grip an outer surface of the surgical tube. The method can also include increasing rigidity at one or more curved portions of the base flange by forming one or more surface protrusions onto an outer surface of the base flange. Also, the one or more surface protrusions can include a first surface protrusion extending from a first curved surface and a second surface protrusion extending from a second curved surface continuous with the first curved surface.

In some instances, a method to manufacture a device operable to secure a surgical tube to a patient includes forming a base flange of a malleable material. The base flange can have a first opening at a first end, the first opening operable to receive the surgical tube. Additionally, the base flange can have a second opening at a second end opposite the first end, the second opening having a diameter greater than the first opening. In some scenarios, the method also include disposing an adhesive onto a bottom surface of the base flange. The adhesive can be operable to adhere the bottom surface to skin of the patient and cause the base flange to form a perimeter around a tube insertion area of the surgical tube extending into the patient.

In some examples, the method also includes increasing rigidity at one or more curved portions of the base flange by forming one or more surface protrusions onto an outer surface of the base flange. The one or more surface protrusions can include a first surface protrusion extending from a first curved portion of the base flange and a second surface protrusion extending from a second curved portion of the base flange continuous with the first curved portion. Additionally, the first curved portion can be convex towards an interior of the base flange, and the second curved portion can be concave towards the interior of the base flange. The second curved portion can also transition into a flat portion of the base flange at the first end.

The foregoing summary is intended to be illustrative and is not meant in a limiting sense. Many features of the examples may be employed with or without reference to other features of any of the examples. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates a perspective view of an example system including a tube placement device for securing a tube to a patient;

FIG. 2 illustrates a front elevation cross-sectional view of an example system including a tube placement device for securing a tube to a patient, which can form at least a portion of the system depicted in FIG. 1;

FIG. 3 illustrates an example method of securing a tube to a patient, which can be performed using the systems depicted in FIGS. 1 and 2;

FIG. 4 illustrates an example method of manufacturing a tube placement device for securing a tube to a patient, which can be performed to form the systems depicted in FIGS. 1 and 2;

FIG. 5 illustrates a side view of an example system including a tube placement device for securing a tube to a patient using an adhesive and/or a fastener, which can form at least a portion of the system depicted in FIG. 1; and

FIG. 6 illustrates an example system including another embodiment of a tube placement device for securing a tube to a patient, which can form at least a portion of the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

### II. General Architecture

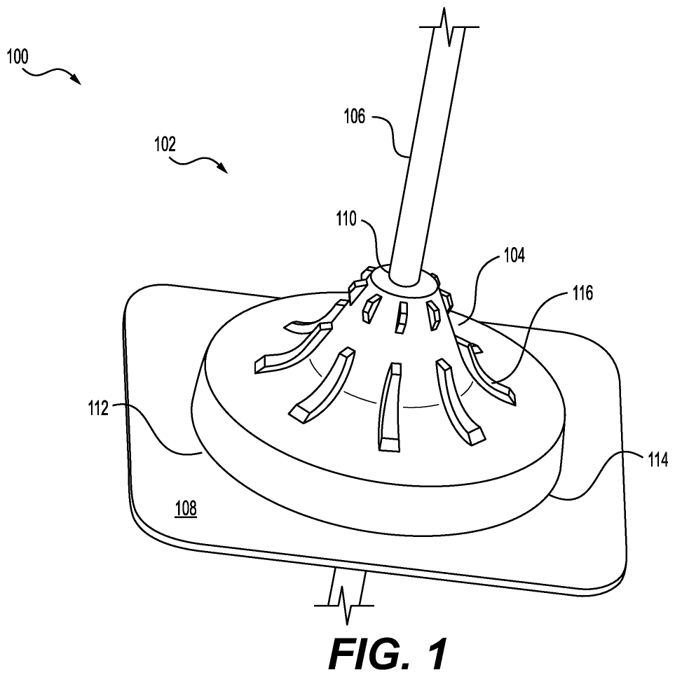

FIG. 1 illustrates an example system 100 of the presently disclosed technology which includes a tube securement device 102. The tube securement device 102 can include a base flange 104 made of a resilient and/or malleable material such as, but not limited to silicone, rubber, or the like. The base flange 104 is operable to circumferentially grip an insertion tube 106 at a surface of a patient's skin 108 in a fluid-occlusive manner.

The tube securement device 102 can have a first opening 110 at a top portion of the base flange 104. The first opening 110 can be operable to receive the insertion tube 106 via a friction-fit engagement. The tube securement device 102 can also have a second opening 112 at a bottom portion of the base flange 104. The second opening 112 can be operable to be secured to the skin 108 of the patient. The first opening 110 can have a smaller diameter than the second opening 112. It is foreseen that the first opening 110 can have a same diameter as the second opening 112 without deviating from scope of the presently disclosed technology. In some instances, an underside interior of the base flange 104 is concave and/or includes a concave curve/contour extending from the first opening 110 to the second opening 112. The base flange 104 can be formed of a wall having a uniform thickness from the first opening 110 to the second opening 112. Moreover, the base flange 104 can be hollow (e.g., with the exterior surface defining an interior space) and/or can include an interior channel or tube connecting the first opening 110 to the second opening 112.

The tube securement device 102 can further include an assembly of the base flange 104 and a fastener 114 such as an adhesive. The fastener 114 can be operable to secure or adhere the tube securement device 102 and/or tubing 106 to the skin 108. The fastener 114 may include an adhesive layer and a selectively-removable cover layer that conceals the adhesive layer and prevents the adhesive layer from deteriorating prior to use of the tube securement device 102 with a patient. It is foreseen that another fastener type other than an adhesive may be used without deviating from the scope of the presently disclosed technology.

The tube securement device 102 can further include one or more secondary fasteners operable to reinforce the friction-fit engagement between the first opening 110 and the insertion tube 106. For instance, it is foreseen that the one or more secondary fasteners may be one or more zip ties, one or more twist ties (or the like), or a combination thereof that wrap circumferentially around the base flange 104 at a designated site to apply additional gripping force between the first opening 110 and the insertion tube 106. In this manner, friction between the first opening 110 and the insertion tube 106 is increased, thereby further preventing any sliding action of the insertion tube 106 relative to the static base flange 104.

It is foreseen that the base flange 104 may include one or more loops to securely receive and position the one or more secondary fastener, e.g., one or more zip tie(s) relative to the base flange 104.

Further to the material of the base flange 104, in the exemplary embodiment, the base flange 104 is made of a silicone material with high elasticity that advantageously permits stretching to accommodate insertion tubes of variable diameters such as, but not limited to diameters ranging from 6 Fr to 40 Fr. In this manner, the tube securement device 102 is advantageously operable to securely receive a wide range of tubes utilized in medicine.

In some instances, the base flange 104 includes one or more surface protrusions 116 extending radially outward from a top surface of the base flange 104 and extending therealong. The one or more protrusions 116 advantageously provide, among other things, structural support and rigidity to help maintain a shape of the base flange 104. The one or more surface protrusions 116 also advantageously provide improved grippability and tactile, sensory feedback to the user of the tube securement device 102, which may facilitate handling, securing of the tube securement device 102 to the patient, and removal of the tube securement device 102 from the patient.

Based on various factors such as, but not limited to the particular application of the tube securement device 102 (e.g., type of tube being supported, portion of the skin to which it is coupled), the tube securement device 102 can have various shapes or sizes. For instance, the base flange 104 can have a circular profile, a triangular profile, a square profile, or the like. Moreover, the one or more surface protrusions 116 can include a single surface protrusions 116, multiple surface protrusions 116, surface protrusions 116 only at one side, surface protrusions 116 extending a partial height of the base flange 104, surface protrusions extending a full height of the base flange 104, combinations thereof, and the like. Additionally, the base flange 104 can have substantially round curves, flat planar surfaces, combinations thereof, and the like.

In some examples, the shapes and/or contours of the tube securement device 102 (e.g., the base flange 104, the one or more surface protrusions 116, etc.) can have an ornamental component and/or can improve an aesthetic look and feel of the tube securement device 102 (e.g., additionally or alternatively to improving a function of the tube securement device 102).

The tube securement device 102 can further include an assembly of the base flange 104 and a fastener 114 such as an adhesive. The fastener 114 is operable to secure or adhere the tube securement device 102 and/or tubing 106 to the skin 108. The fastener 114 may include an adhesive layer and a selectively-removable cover layer that conceals the adhesive layer and prevents the adhesive layer from deteriorating prior to use of the tube securement device 102 with a patient. It is foreseen that another fastener type other than an adhesive may be used without deviating from the scope of the presently disclosed technology.

The tube securement device 102 can further include one or more secondary fasteners operable to reinforce the friction-fit engagement between the first opening 110 and the insertion tube 106. For instance, it is foreseen that the one or more secondary fasteners may be one or more zip ties, one or more twist ties (or the like), or a combination thereof that wrap circumferentially around the base flange 104 at a designated site to apply additional gripping force between the first opening 110 and the insertion tube 106. In this manner, friction between the first opening 110 and the insertion tube 106 is increased, thereby further preventing any sliding action of the insertion tube 106 relative to the static base flange 104.

It is foreseen that the base flange 104 may include one or more loops to securely receive and position the one or more secondary fastener, e.g., one or more zip tie(s) relative to the base flange 104.

Further to the material of the base flange 104, in the exemplary embodiment, the base flange 104 is made of a silicone material with high elasticity that advantageously permits stretching to accommodate insertion tubes of variable diameters such as, but not limited to diameters ranging from 6 Fr to 40 Fr. In this manner, the tube securement device 102 is advantageously operable to securely receive a wide range of tubes utilized in medicine.

In some instances, the base flange 104 includes one or more surface protrusions 116 extending radially outward from a top surface of the base flange 104 and extending therealong. The one or more protrusions 116 advantageously provide, among other things, structural support and rigidity to help maintain a shape of the base flange 104. The one or more surface protrusions 116 also advantageously provide improved grippability and tactile, sensory feedback to the user of the tube securement device 102, which may facilitate handling, securing of the tube securement device 102 to the patient, and removal of the tube securement device 102 from the patient. Furthermore, the surface protrusion(s) 116 can define a recessed area for securing one or more fasteners (e.g., zip-ties) arounds the upper portion of the base flange, thus tightening the first opening 110 around the insertion tube 106, as discussed in greater detail below.

Based on various factors such as, but not limited to the particular application of the tube securement device 102 (e.g., type of tube being supported, portion of the skin to which it is coupled), the tube securement device 102 can have various shapes or sizes. For instance, the base flange 104 can have a circular profile, a triangular profile, a square profile, or the like. Moreover, the one or more surface protrusions 116 can include a single surface protrusion 116, multiple surface protrusions 116, surface protrusions 116 only at one side, surface protrusions 116 extending a partial height of the base flange 104, surface protrusions extending a full height of the base flange 104, combinations thereof, and the like. Additionally, the base flange 104 can have substantially round curves, flat planar surfaces, combinations thereof, and the like.

In some examples, the shapes and/or contours of the tube securement device 102 (e.g., the base flange 104, the one or more surface protrusions 116, etc.) can have an ornamental component and/or can improve an aesthetic look and feel of the tube securement device 102 (e.g., additionally or alternatively to improving a function of the tube securement device 102).

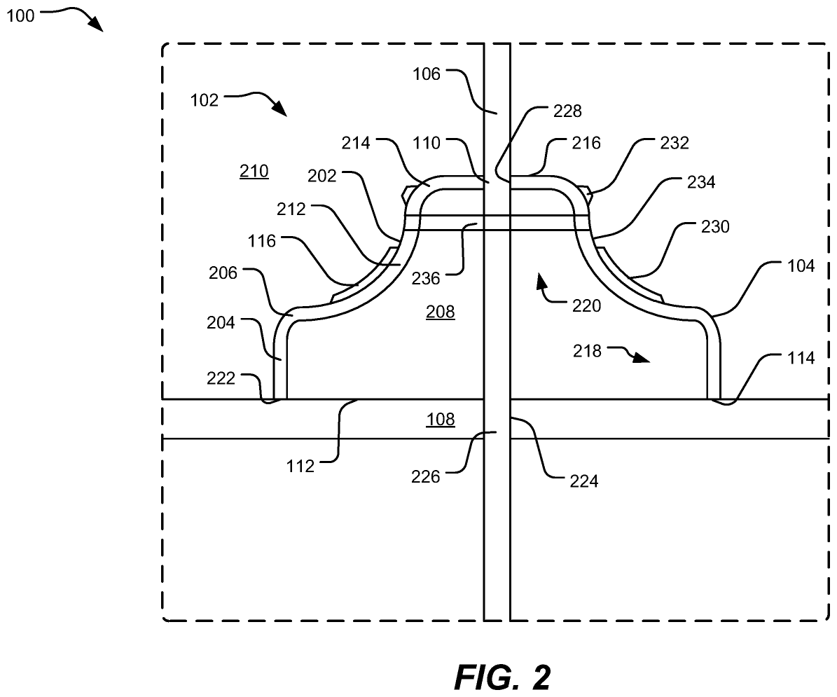

FIG. 2 illustrates a cross-sectional view of the system 100 depicting the tube securement device 102 gripping the insertion tube 106 and securing the insertion tube 106 to the skin 108.

As shown in FIG. 2, the base flange 104 can have a partially flexible, semi-rigid, and/or malleable outer shell 202 with one or more curves forming a multi-curved cross sectional profile. For instance, the outer shell 202 can include a sidewall 204 extending substantially perpendicularly from the skin 108 and having a first curve 206 (e.g., bend, corner, and/or contour), which can be concave towards an interior 208 defined by the rigid outer shell 202 (e.g., convex towards an exterior 210). The first curve 206 can transition into a second curve 212 that is convex towards the interior 208 (e.g., concave towards the exterior 210). The second curve 212 can transition into a third curve 214 which is concave towards the interior 208 (e.g., convex towards the exterior 210). The third curve 214 can transition into a straight or substantially flat portion 216 which includes, centrally located, the first opening 110. The arrangement of the first curve 206, the second curve 212, and/or the third curve 214 with the straight or substantially flat portion 216 can form a profile for the tube securement device 102 that has a wider lower body 218 (e.g., proximal to the skin 108) that transitions into a narrower upper body 220 (e.g., distal from the skin).

Accordingly, in some examples, the attachment surfaces of the tube securement device 102 can form smooth and consistent attachments with the surfaces of the insertion tube 106 and the skin 108. For instance, a skin adjoining perimeter surface 222 of the lower body 218 (e.g., defining the perimeter around the second opening 112 and around the insertion tube 106 at the skin interface), can be a flat surface that forms flush contact with the skin 108. The flush contact can be secured to the skin with the one or more fastener(s) 114 (e.g., adhesive, tape, thread, etc.). Additionally or alternatively, one or more tube fastener(s) 224 (e.g., adhesive, tape, thread, etc.) can couple the insertion tube 106 to the skin 108 at a skin opening 226 by adhering an outer surface of the insertion tube 106 to an inner dermal surface formed at the puncture or cut into the skin 108. Moreover, an inner surface 228 of the first opening 110 can be flush with and/or contact the outer surface of the insertion tube 106, and can be secured in place with the tube fastener(s) 224 (e.g., the same or different than that used to adhere to the skin 108).

Furthermore, in some instances, the one or more surface protrusions 116 can provide structural rigidity to the one or more curves (e.g., the first curve 206, the second curve 212, and/or the third curve 214). An elongated protrusion 230 can extend from the second curve 212 and/or can provide structural support to the second curve 212. Additionally or alternatively, a smaller upper curve protrusion 232 can protrude from the third curve 214 to provide structural support for the third curve 214 and/or to maintain the inner surface 228 of the first opening 110 flush with the outer surface of the insertion tube 106. These various components of the tube securement device 102 can provide secure attachments for the insertion tube 106 to the skin 108, which stay in place even when the insertion tube 106 is bumped and moved in directions non-perpendicular to the skin. Moreover, the insertion tube 106 can prevent some of the movement of the insertion tube 106 relative to the skin 108 at locations proximate to or at the skin opening 226, and/or in the interior 208 defined by the outer shell 202. In some scenarios, a recessed area 234 is defined between the upper curve protrusion 232 and the elongated protrusion 230, which can receive a fastener 236 (e.g., a zip-tie, a metal strip, etc.), and/or prevent the fastener 236 from sliding up or down the base flange 104. In other words, the recessed area 234 can form a seat for the fastener 236 to prevent the fastener 236 from slipping off the end of the base flange 104.

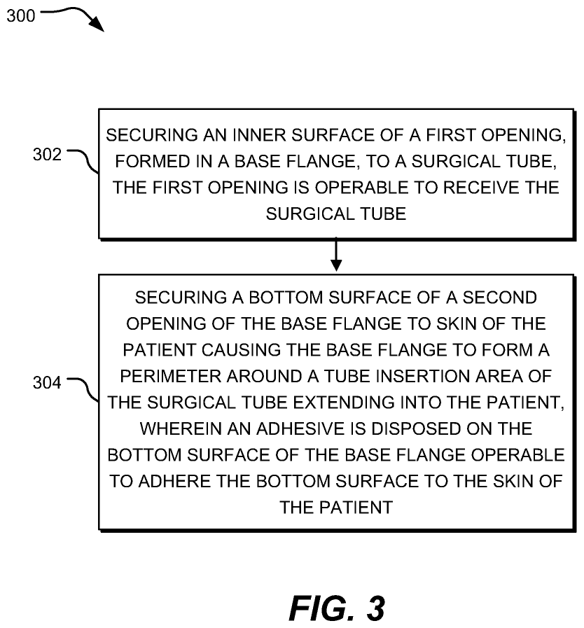

FIG. 3 illustrates an example method 300 for securing a tube securement device tube securement device 102, which can be performed by or form a portion of the system 100, for instance, by medical personnel and/or a receiver of the tube securement device 102.

In some instances, at operation 302, the method 300 can secure an inner surface of a first opening, formed in an outer shell of a base flange, to a surgical tube, the first opening being operable to receive the surgical tube. At operation 304, the method 300 can secure a bottom surface of a second opening of the base flange to skin of the patient causing the base flange to form a perimeter around a tube insertion area of the surgical tube extending into the patient, wherein an adhesive is disposed on the bottom surface of the base flange operable to adhere the bottom surface to the skin of the patient.

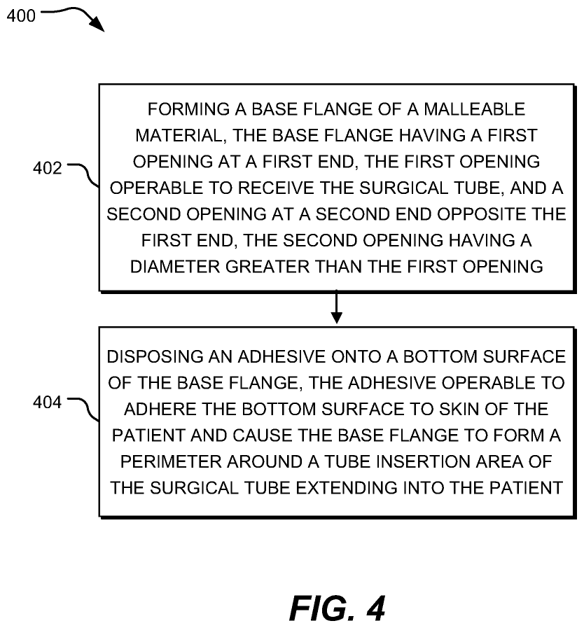

FIG. 4 illustrates an example method 400 for manufacturing a tube securement device 102, which can be performed or form a portion of the system 100.

In some examples, at operation 402, the method 400 forms a base flange using a malleable material with a circular profile and having: a first opening at a first end, the first opening operable to receive the surgical tube, and a second opening at a second end opposite the first end, the second opening having a diameter greater than the first opening. At operation 404, the method 400 disposes an adhesive onto a bottom surface of the base flange, the adhesive operable to adhere the bottom surface to skin of the patient and cause the base flange to form a perimeter around a tube insertion area of the surgical tube extending into the patient.

It is to be understood that the specific order or hierarchy of steps in the method(s) depicted in FIG. 4 and throughout this disclosure are instances of example approaches and can be rearranged while remaining within the disclosed subject matter. For instance, any of the operations depicted in FIG. 4 and throughout this disclosure may be omitted, repeated, performed in parallel, performed in a different order, and/or combined with any other of the operations depicted in FIG. 4 and throughout this disclosure.

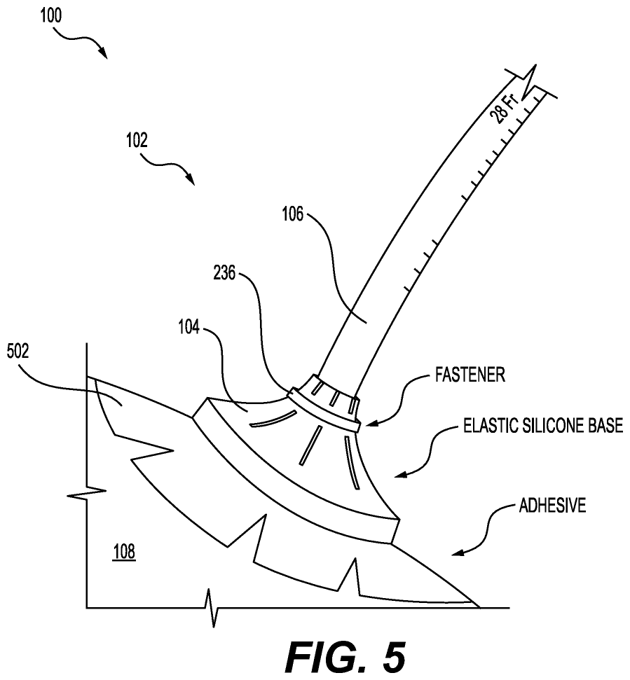

FIG. 5 illustrates a side view of the system 100 depicting the tube securement device 102 gripping the insertion tube 106 and securing the insertion tube 106 to the skin 108. As shown in FIG. 5, the tube securement device can omit the surface protrusions 106.

Furthermore, in some instances, an adhesive layer 502 can be disposed on the skin 108 below the base flange 104. Additionally, the fastener 236 can be disposed around the recessed area 234 between the second curve 212 and the third curve 214. The recessed area 234 can include an indent or other physical feature at least partially or fully wrapping around a circumference of the base flange 104 to form a seat for the fastener 236.

In some examples, the base flange 104 can be formed of either thermoplastic elastomer or silicone with high elongation at break to elastically receive tubes of varying diameter, which can far exceed the baseline diameter of the first hole 110 in the base flange 104. The holes 110 of the base flange 104 can stretch to accommodate large tubes. The surface of the base flange 104 can have a high coefficient of friction, providing increased grippability. In addition, as discussed herein, the recessed area 234 can be used to place a zip tie or other type of fastener 236 tightly and circumferentially around the base flange 104, creating additional normal force against the insertion tube 106, thereby increasing a friction force holding the insertion tube 106 in place.

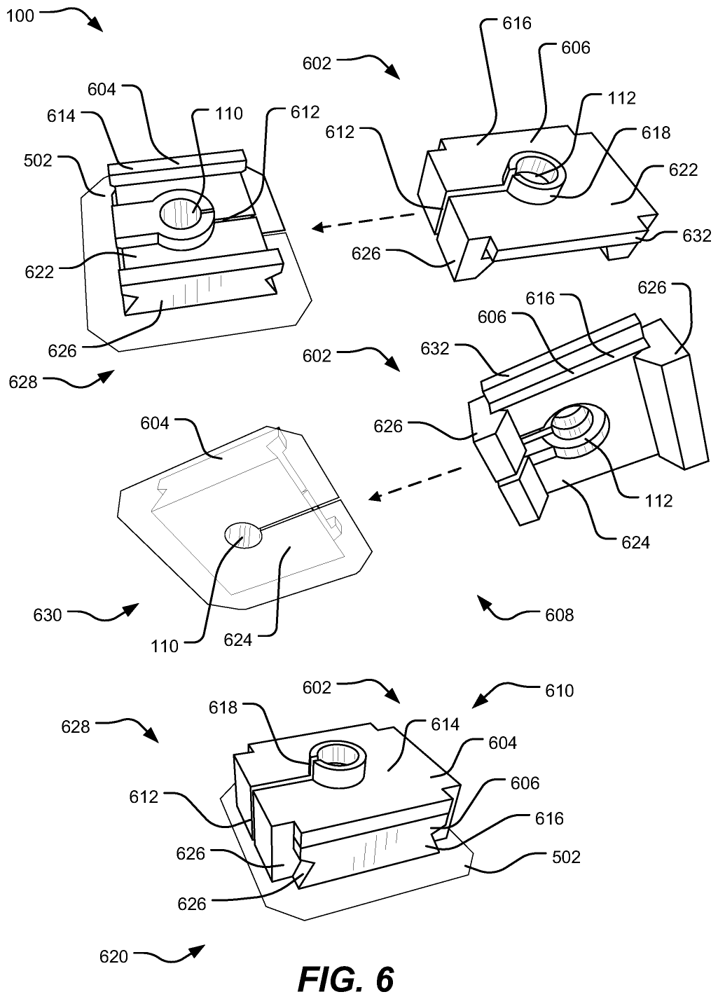

FIG. 6 illustrates an example of the system 100 including another embodiment 602 of the tube securement device 102. This embodiment 602 can include a first portion 604 which mates with a second portion 606 to transition the tube securement device 102 between an unmated position 608 and a mated position 610, as discussed in greater detail below.

In some examples, issues can arise if the tube securement device 102 must be placed on the chest tube 106 prior to chest tube insertion into the body. Furthermore, if the tube securement device 102 needs to be removed/replaced, it can be difficult to take off in scenarios involving full circumferential wrapping around the tube. As such, some examples of the tube securement device 102 may be disposable and/or may include a step of being cut, damaged, and/or replaced when the tube securement device 102 needs to be removed.

To address these issues, in some examples, the tube securement device 102 can include one or more slits 612, which can operate as attachment slits or removal slits for sliding the tube securement device 102 onto and/or off of the insertion tube 106, which can occur after the insertion tube 106 has been inserted into the body. Furthermore, the embodiment 602 of the tube securement device 102 depicted in FIG. 6 can include two separate parts that, when combined, achieve a fluid-occlusive seal. The two parts (e.g., the first portion 604 and the second portion 606) can be wrapped onto and off of the tube while the tube remains in the body. These components can provide an installation procedure after the tube is inserted in the body, can make removal/replacement simpler, and/or can provide for readjustments of the tube securement device 102.

In some scenarios, the first portion 604 of the tube securement device 102 can be a bottom piece 614, which can comprise an elastic, silicone, or other type of malleable component with a skin adhesive layer 502. This bottom piece 614 can have a slit 612 in the side allowing it to be spread open to wrap onto the insertion tube 106, placing the insertion tube 106 within the central hole of the bottom piece 614. The bottom piece 614 can then be adhered to the patient's skin using the adhesive. The top piece 616 (e.g., the second portion 606) can be separately wrapped around the tube, then pressed on top of the bottom piece 614. The two parts can be held together by the depicted geometry, such that teeth or other protrusion(s) on the bottom piece 614 and/or the top piece 616 engage with each other and prevent separation. The top piece 616 can also include a neck 618 at the top to receive a fastener 236 such as a zip-tie to strongly secure the mated assembly 620 to the tube 106 (e.g., to prevent the tube from sliding within the tube securement device 102).

In some examples, the first portion 604 and/or the second portion 606 can have additional corresponding shapes and/or features to improve the attachment and removal processes for the tube securement device 102. For example, the first portion 604 and/or the second portion 606 can have an at least partially square, rectangle, or circular profile, which can have matching curves/lines to mate the two components together. The first portion 604 and the second portion 606 can include other protrusions, indents, keyed portions, flexible hinges, or other mating features such that the first portion 604 can engage the second portion 606 and hold the components in the removable mated position 610. In some examples, a top surface 622 of the tube securement device 102 can define the first opening 110, while an undersurface 624 or inner surface of the tube securement device 102 (e.g., at the bottom piece 614 or the top piece 616) can define the second opening 112. Additionally or alternatively the second opening 112 of the tube securement device 102 can be defined by one or more sidewalls 626 extending down from the first portion 604 and/or the second portion 606. Moreover, the second opening 112 can be defined into a surface of the bottom piece 614, while the first opening 110 can be formed into a planar surface of the top piece 616. FIG. 6 depicts a perspective top view 628 of the two components and a perspective bottom view 630 of the components, showing the first opening 110 and the second opening 112 formed into the bottom piece 614 and the top piece 616, respectively. The first portion 604 and/or the second portion 606 can include one or more lips 632 and/or corresponding channels for guiding a sliding mating motion and/or for further locking the two components together. The first opening 110 can have a smaller diameter than the second opening 112 or the diameters can be the same. Additionally or alternatively, the bottom piece 614 and the top piece 616 can include one or more slits 612 oriented in different directions (e.g., at 180° or 90° differences) to further secure the insertion tube 106 in place when the first portion 604 and the first portion second portion 606 are in the mated position 610.

While the presently disclosed technology has been described with reference to various implementations, it will be understood that these implementations are illustrative and that the scope of the presently disclosed technology is not limited to them. Many variations, modifications, additions, and improvements are possible. More generally, implementations in accordance with the presently disclosed technology have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A device operable to secure a surgical tube to a patient, the device comprising:
at least one of a first portion or a second portion having:
an opening at a first end, the opening operable to receive the surgical tube and friction-grip the surgical tube, and
one or more slits formed into a side of at least one of the first portion or the second portion; and
an adhesive disposed on a bottom surface at a second end of at least one of the first portion or the second portion, the adhesive operable to adhere the bottom surface to skin of the patient substantially surrounding a tube insertion area of the surgical tube into the patient.

2. The device of claim 1,
wherein,
at least one of the first portion or the second portion are formed of a malleable material.

3. The device of claim 1,
wherein,
the first portion is a bottom piece of the device, and the second portion is a top piece of the device that mates with the bottom piece.

4. The device of claim 3, further comprising:
a first slit disposed on the bottom piece and a second slit disposed on the top piece configured to align in a different direction than the first slit when the top piece mates with the bottom piece.

5. The device of claim 4,
wherein,
the first slit is directed 180° or 90° relative to the second slit.

6. The device of claim 1, further including,
one or more curved portions include a first curved portion directed concavely toward an interior of a base flange and forming a transition between a sidewall of the base flange and a second curved portion of the base flange, and
one or more surface protrusions including an elongated protrusion at the second curved portion.

7. The device of claim 1, further comprising;
one or more protrusions extending from the second portion configured to mate with a side of the first portion.

8. The device of claim 1, further including an opening neck extending from a planar surface of the second portion.

9. The device of claim 1,
wherein,
the device has an upper portion including a first opening, and a lower portion including a second opening, and
the first opening is narrower than at least a portion of the second opening.

10. A method of using a device operable to secure a surgical tube to a patient, the method comprising:
securing an inner surface of a first opening formed in a base flange to a surgical tube, the first opening operable to receive the surgical tube; and
securing a bottom surface forming a perimeter of a second opening of the base flange to skin of the patient causing the base flange to form the perimeter around a tube insertion area of the surgical tube extending into the patient, wherein an adhesive is disposed on the bottom surface of the base flange operable to adhere the bottom surface to the skin of the patient.

11. The method of claim 10,
wherein,
the first opening is at a first end of the base flange, and
the second opening is at a second end of the base flange opposite the first end.

12. The method of claim 10,
wherein,
the second opening has a diameter greater than the first opening.

13. The method of claim 10,
wherein,
securing the inner surface of the first opening to the surgical tube includes using a friction-fit to grip an outer surface of the surgical tube.

14. The method of claim 10,
wherein,
the base flange includes an outer surface with one or more surface protrusions operable to increase rigidity at one or more curved portions of the base flange.

15. The method of claim 14,
wherein,
the one or more surface protrusions includes a first surface protrusion extending from a first curved surface and a second surface protrusion extending from a second curved surface continuous with the first curved surface.

16. A method of manufacturing a device operable to secure a surgical tube to a patient, the method comprising:
forming a base flange of a malleable material, the base flange having:
a first opening at a first end, the first opening operable to receive the surgical tube, and
a second opening at a second end opposite the first end, the second opening having a diameter greater than the first opening; and
disposing an adhesive onto a bottom surface of the base flange, the adhesive operable to adhere the bottom surface to skin of the patient and cause the base flange to form a perimeter around a tube insertion area of the surgical tube extending into the patient.

17. The method of claim 16, further comprising:
increasing rigidity at one or more curved portions of the base flange by forming one or more surface protrusions onto an outer surface of the base flange.

18. The method of claim 17,
wherein,
the one or more surface protrusions includes a first surface protrusion extending from a first curved portion of the base flange and a second surface protrusion extending from a second curved portion of the base flange continuous with the first curved portion.

19. The method of claim 18,
wherein,
the first curved portion is convex towards an interior of the base flange, and
the second curved portion is concave towards the interior of the base flange.

20. The method of claim 19,
wherein,
the second curved portion transitions into a flat portion of the base flange at the first end.
